(19) United States
(12) Patent Application Publication
(10) Pub. No.: US 2005/0101017 A1
(43) Pub. Date: May 12, 2005

(54) Method of improving gene targeting using a ubiquitin promoter

(76) Inventors: Wojtek Auerbach, Ridgewood, NJ (US); David Frendewey, New York, NY (US); Andrew J. Murphy, Croton-on-Hudson, NY (US); David M. Valenzuela, Yorktown Heights, NY (US)

Correspondence Address: REGENERON PHARMACEUTICALS, INC, 777 OLD SAW MILL RIVER ROAD, TARRYTOWN, NY, 10591, US

(21) Appl. No.: 10/705,432

(22) Filed: Nov. 10, 2003

(51) Int. Cl. C12N 5/06; C12N 15/85

(52) U.S. Cl. 435/455; 435/354

(57) ABSTRACT

Methods of improving gene targeting comprising using a ubiquitin promoter to drive drug resistance gene expression are provided. Such improvements include increasing ES cell colony survival and targeting frequency.

### FIELD OF THE INVENTION

The field of the invention is related to a method for improving gene targeting comprising using a ubiquitin promoter to drive expression of a drug resistance gene.

### DESCRIPTION OF RELATED ART

Schorpp, et al., report that the ubiquitin C promoter directs high expression of transgenes in mice (Nucleic Acids Research, 1996, 24(9):1787-1788).

### BRIEF SUMMARY OF THE INVENTION

The method of the invention is based in part on the finding that the use of a ubiquitin promoter to drive expression of a drug resistance gene in gene targeting experiments results in an increase in the number of transfected embryonic stem (ES) cells surviving drug selection. In addition, the use of a ubiquitin promoter was found to drive expression of a drug resistance gene in gene targeting experiments increases the overall gene targeting frequency in ES cells.

Accordingly, a first aspect of the invention is a method of increasing the number of ES cell colonies exhibiting drug resistance to a selection agent comprising introducing into the ES cells an exogenous DNA comprising a ubiquitin promoter driving expression of a drug resistance gene.

A related second aspect of the invention is a method of increasing the targeting efficiency of a targeting vector introduced into ES cells comprising introducing into the ES cells a targeting vector comprising a ubiquitin promoter driving expression of a drug resistance gene.

In one embodiment, the ubiqutin promoter is a ubiquitin C (UbC) promoter. In a specific embodiment, the UbC promoter is a human, mouse, or rat UbC promoter. In separate embodiments, the human UbC promoter has the sequence set forth in SEQ ID NO: 1, the mouse UbC promoter has the sequence set forth in SEQ ID NO: 2, and the rat UbC promoter has the sequence set forth in SEQ ID NO: 3.

Other embodiments are ones in which the ubiquitin promoter is a promoter selected from the promoters of the genes set forth in Table 1 below. Such promoters can be derived from ubiquitin C genes of various species including, but not limited to human, mouse, rat, A. thaliana, C. elegans, and D. melanogaster; they can be derived from ubiguitin genes other than ubiquitin C of various species including, but not limited to, human, mouse, rat, A. thaliana, C. elegans, and D. melanogaster, and B. Taurus; or they can be derived from ubiquitin-like genes of various species including, but not limited to, human, mouse, rat, A. thaliana, C. elegans, and D. melanogaster.

In one embodiment, the ES cells are mammalian ES cells. In a particular embodiment, the mammalian ES cells are rat, mouse, rabbit, cat, dog, cow, sheep, goat, pig, horse, or monkey ES cells. In a specific embodiment, the ES cells are mouse ES cells.

In one particular embodiment of the invention, the drug resistance gene is the neomycin-resistance gene (neo^{r}). In another particular embodiment, the drug resistance gene is the hygromycin-resistant gene (hyg^{r}). In still another embodiment, the drug resistance gene is the puromycin-resistance gene (puro^{r}). Other embodiments are ones in which the drug resistance genes are negative selection genes such as herpes simplex virus-thymidine kinase (HSV-tk) and fusions of tk with neo^{r}, hyg^{r}, or puro^{r}.

Other objects and advantages will become apparent from a review of the ensuing detailed description.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned are incorporated herein by reference in their entirety.

Definitions

By “targeting vector” as used herein is meant a DNA construct that contains sequences “homologous” to endogenous chromosomal nucleic acid sequences flanking a desired genetic modification(s). The flanking homologous sequences, referred to as “homology arms”, direct the targeting vector to a specific chromosomal location within the genome by virtue of the homology that exists between the homology arms and the corresponding endogenous sequence and effect a desired genetic modification by a process referred to as “homologous recombination”. By “homologous” as used herein is meant two or more nucleic acid sequences that are either identical or similar enough that they are able to hybridize to each other or undergo intermolecular exchange.

By “gene targeting” as used herein is meant the modification of an endogenous chromosomal locus by the insertion into, deletion of, or replacement of the endogenous sequence, or a portion thereof, via homologous recombination using a targeting vector.

By “gene knockout” as used herein is meant a genetic modification resulting from the disruption of the genetic information encoded in a chromosomal locus. By “gene knockin” as used herein is meant a genetic modification resulting from the replacement of the genetic information encoded in a chromosomal locus with a different DNA sequence. By “knockout organism” as used herein is meant an organism in which a significant proportion of the organism's cells harbor a gene knockout. By “knockin organism” as used herein is meant an organism in which a significant proportion of the organism's cells harbor a gene knockin.

By “drug resistance gene” as used herein is meant a gene whose expression allows for the survival of rare transfected cells expressing the gene from the majority of treated cells in the population. Such drug resistance genes include, but are not limited to, neo^{r}, hyg^{r}, or puro^{r} or negative selection genes such as HSV-tk and fusions of tk with neo^{r}, hyg^{r}, or puro^{r}.

An “ES cell” as used herein is meant to mean an embryonic stem cell. This cell is usually derived from the inner cell mass of a blastocyst-stage embryo. By “blastocyst” is meant the mammalian conceptus in the post-morula stage, consisting of the trophoblast and an inner cell mass. An “ES cell clone” as used herein is a subpopulation of cells derived from a single cell of the ES cell population following introduction of DNA and subsequent selection.

By “non-human organism” as used herein is meant an organism that is not normally accepted by the public as being human.

By “mutating” or “mutation” as used herein is meant any change including, but not limited to, additions, deletions, substitutions or other modifications of one or more nucleotides in a DNA sequence.

By “recombinase” as used herein is meant an enzyme that recognizes specific nucleotide sequences termed “recombination sites” or “site-specific recombination sites” and that catalyzes recombination of DNA between these sites. Recombinases are able to either delete sequences between the site-specific recombination sites if the sites are oriented in the same direction with respect to one another or invert the sequences between the site-specific recombination sites if the sites are oriented in opposite directions with respect to one another.

By “polyadenylation signal sequence” or “pA” as used herein is meant a nucleotide sequence that is recognized by the RNA processing machinery that forms the 3′ ends of mRNA by cleavage of the nascent transcript followed by polymerization of adenosine nucleotides to the cleaved end.

“Ubiquitin promoter” as used herein means the region of genomic DNA up to 5000 base pairs (bp) upstream from either the start codon, or a mapped transcriptional start site, of a ubiquitin, or ubiquitin-like, gene.

General Description

Applicants have discovered that using a ubiquitin promoter to drive expression of a drug resistance gene such as neo^{r} as part of a gene targeting vector results in an increase in the number of ES cell colonies exhibiting drug resistance to a selection agent following introduction of the targeting vector. Applicants have also discovered that use of a ubiquitin promoter to drive drug resistance gene expression also increases the overall targeting efficiency in ES cells.

Ubiquitin Genes and Promoters

Ubiquitin is an abundant 76 amino acid polypeptide found in all eukaryotic cells. There are several different genes that encode ubiquitin and their homology at the amino acid level is quite high. For example, human and mouse have many different genes encoding ubiquitin, each located at a different chromosomal locus. Functionally, all ubiquitin genes are critical players in the ubiquitin-dependent proteolytic machinery of the cell. Each ubiquitin gene is associated with a promoter that drives its expression. A ubiquitin promoter is the region of genomic DNA up to 5000 bp upstream from either the start codon, or a mapped transcriptional start site, of a ubiquitin, or ubiquitin-like, gene. Ubiquitin genes and their promoters that have been identified so far include, but are not limited to, those set forth in Table 1 below. One of skill in the art will recognize that any ubiquitin promoter may be amendable to the methods of the invention.

TABLE 1

| | | | |
|---|---|---|---|
| Species | Gene name | Acc # | gi# |

Ubiquitin C genes:

| | | | |
|---|---|---|---|
| human | UbC | NM_021009 | 34304116 |
| Mouse | UbC | XM_287520 | 28548342 |
| rat | UbC | NM_017314 | 8394501 |
| A. thaliana | UBQ8 | NM_111814 | 18398637 |
| C. elegans | ubq-1 | NM_171139 | 25151715 |
| D. melanogaster | Ubi-p63E | NM_168043 | 24657013 |

Other Ubiquitin genes:

| | | | |
|---|---|---|---|
| human | UBA52 | NM_003333 | 15451941 |
| mouse | UBA52 | XM_134243 | 28495015 |
| rat | UBA52 | NM_031687 | 13928951 |
| A. thaliana | UBQ1 | NM_115119 | 18409638 |
| C. elegans | ubq-2 | NM_067294 | 17554757 |
| D. melanogaster | Ubi-f52 | NM_057428 | 24581598 |
| human | UbB | NM_018955 | 22538474 |
| Mouse | UbB | NM_011664 | 6755918 |
| Rat | UbB | NM_138895 | 20302084 |
| B. taurus | UbB | NM_174133 | 27806504 |
| human | UbD | NM_006398 | 5454143 |
| mouse | UbD | NM_023137 | 13194204 |
| rat | UbD | NM_053299 | 1675799 |
| human | UBA80 | NM_002954 | 27436941 |
| mouse | Rps27a | NM_024277 | 13195689 |
| rat | Rps27a | NM_031113 | 13592076 |
| A. thaliana | UBQ5 | NM_116090 | 18412305 |
| D. melanogaster | Ip259 | NM_058031 | 28574121 |

Ubiquitin-like genes:

| | | | |
|---|---|---|---|
| human | NEDD8 | NM_006156 | 5453759 |
| mouse | NEDD8 | NM_008683 | 6679033 |
| rat | NEDD8 | NM_138878 | 20302050 |
| C. elegans | NEDD8 | NM_060316 | 17507350 |
| D. melanogaster | NEDD8 | NM_136075 | 24585073 |
| human | UBL4 | NM_014235 | 7657666 |
| mouse | UBL4 | NM_145405 | 21703809 |
| rat | UBL4 | XM_215228 | 27682126 |
| human | Sumo (ubl1) | NM_003352 | 20127433 |
| mouse | Sumo (ubl1) | NM_009460 | 6678488 |
| rat | Sumo (ubl1) | XM_217413 | 27683946 |
| human | Elongin-B | NM_007108 | 6005889 |
| mouse | Elongin-B | NM_026305 | 3385799 |
| rat | Elongin-B | NM_031129 | 13592104 |
| D. melanogaster | Elongin-B | NM_079692 | 24648446 |
| human | PARK2 | NM_004562 | 4758883 |
| mouse | PARK2 | NM_016694 | 7710077 |
| rat | PARK2 | NM_020093 | 11464986 |
| human | UBL3 | NM_007106 | 6005927 |
| mouse | UBL3 | NM_011908 | 6755924 |
| rat | UBL3 | XM_237860 | 27691977 |
| D. melanogaster | UBL3 | NM_132855 | 24642320 |

Drug Resistance Genes

Many different drug resistance genes are known in the art and are useful in practicing the invention. Non-limiting examples include neomycin phosphotransferase (neo^{r}), hygromycin B phosphotransferase (hyg^{r}), puromycin-N-acetyltansferase (puro^{r}), blasticidin S deaminase (bsr^{r}), xanthine/guanine phosphoribosyl transferase (gpt), Herpes simplex virus thymidine kinase (HSV-tk), and fusions of tk with neo^{r}, hyg^{r} or puro^{r}. Suitable selection agents for the drug resistance genes include G418 (with neo^{r}), puromycin (with puro^{r}), hygromycin B (with hyg^{r}), blasticidin S (with bsr^{r}), mycophenolic acid and 6-thioxanthine (with gpt), and ganciclovir or 1(2′-deoxy-2′-fluoro-β-D-arabinofuranosyl)-5-iodouracil (FIAU) (with HSV-tk). Other selection agents include toxins such as diptheria toxin A fragment (DTA).

Nucleic Acid Constructs

The techniques used to obtain the components of the targeting vectors and to construct the targeting vectors described herein are standard molecular biology techniques well known to the skilled artisan (see e.g., Sambrook, J. and Russell, Molecular Cloning: A Laboratory Manual, Third Edition, Vols 1, 2, and 3, 2001). Any of the methods known to one skilled in the art for the insertion of DNA fragments into a vector may be used to construct the targeting vectors of the invention. One standard molecular biology technique useful in constructing the targeting vectors containing a ubiquitin promoter driving expression of a drug resistance gene is bacterial homologous recombination. For a detailed description of how one might construct such targeting vectors, see U.S. Pat. No. 6,586,251, in the name of Regeneron Pharmaceuticals Inc. and Valenzuela et al. (2003) Nature Biotechnology 21(6):652-659, each of which are incorporated herein by reference. All DNA sequencing is done by standard techniques using an ABI 373A DNA sequencer and Taq Dideoxy Terminator Cycle Sequencing Kit (Applied Biosystems, Inc., Foster City, Calif.).

The targeting vectors containing ubiquitin promoters driving expression of a drug resistance gene that are useful in practicing the methods of the invention can be constructed in a variety of ways. While any ubiquitin promoter may be suitable for use in the methods of the invention, a ubiquitin promoter that has been characterized is most useful. An example of a suitable characterized ubiquitin promoter is that for ubiquitin C (UbC). Preferably, a human UbC promoter is used. Once a ubiquitin promoter is chosen, it can be incorporated into a targeting vector such that it drives expression of a drug resistance gene. In addition, any number of exogenous DNA sequences may be included in the targeting vector including, but not limited to, site-specific recombination sites (e.g. loxP sites or FRT sites). It is also possible to include other exogenous DNA sequences in association with the ubiquitin promoter such as pA sequences as well as other regulatory sequences capable of turning on, turning off, enhancing, down-regulating or otherwise modulating gene expression.

ES Cell Colony Number and Targeting Efficiency

The number of correctly targeted ES cell clones is a multiple of the number of drug resistant ES cell colonies and the frequency of targeting events. Therefore, it is desirable to increase the number of drug resistant ES cell colonies in order to increase the probability of obtaining correctly targeted ES cell clones.

Targeting vectors introduced into cells are subject to two competing events: homologous recombination at the target chromosomal locus or non-homologous random integration into the genome. Because random integration tends to predominate, the use of targeting vectors that increase the chance of correct modification of the target locus offer significant advantages. Two major variables are commonly considered to influence targeting frequency: first, poorly defined characteristics of the target locus, such as its DNA sequence or chromatin structure; and second, the length and degree of homology between sequences in the targeting vector and those at the target locus. In addition, many enrichment schemes can be utilized to select against randomly integrated clones in culture, such as negative selection against non-homologous recombination events. As described below, changing the promoter driving or controlling expression of a drug resistance gene to a ubiquitin promoter significantly increases the ratio of targeted to total drug resistant clones.

Mammalian Cells

All mammalian cells are amendable to the methods of the invention because all mammalian cells contain DNA and the enzymatic machinery that facilitates homologous recombination. Examples of preferred mammalian cells useful in practicing the methods of the invention are those derived from rat, mouse, rabbit, cat, dog, cow, sheep, goat, pig, horse, or monkey. Preferred mammalian cells useful for practicing the invention are stem cells, including ES cells. While stem cells, including ES cells, from all species are suitable, the most preferred stem cells, including ES cells, are rodent cells. In particular, mouse stem cells, especially mouse ES cells, are useful for practicing the methods of the invention.

Introduction of DNA into Mammalian Cells

The DNA, including DNA targeting vectors and other types of DNA such as linear segments of DNA, useful in practicing the methods of the invention can be introduced into mammalian cells such as ES cells using standard methodologies such as transfection mediated by calcium phosphate, lipids, or electroporation (Sambrook, J. and Russell, Molecular Cloning: A Laboratory Manual, Third Edition, Vols 1, 2, and 3, 2001). The cells in which the DNA has been introduced successfully can be selected by exposure to any number of selection agents, depending on the selectable marker gene that has been engineered into the introduced DNA. As a non-limiting example, if the selectable marker gene is the neo^{r} gene, then cells that have taken up the DNA can be selected in media containing G418; cells that do not have the DNA will die whereas cells that have taken up the DNA will survive. Other suitable selectable markers include any agent that has activity in mammalian cells such as hygromycin B as well as other agents familiar to those skilled in the art.

Identification of Genetically Mutated and/or Modified Mammalian Cells

Mammalian cells, for example ES cells, that have been successfully genetically modified by the methods of the invention can be identified using a variety of approaches and assays. Such approaches and assays can include but are not limited to: (a) Southern blotting, (b) long PCR, (c) quantitative PCR using TaqMan® (see Lie and Petropoulos, Curr Opin Biotechnol, 9:43-8, 1998, molecular beacons (see Tan et al. (2000) Chemistry, 6:1107-11) SYBR green, LUX primers (Invitrogen), and qZyme® (BD Bioscience); (d) fluorescence in situ hybridization (FISH) (see Laan et al. (1995) Hum Genet 96:275-80) or comparative genomic hybridization (CGH) (see for example Forozan et al. (1997) Trends Genet 13:405-9); (e) isothermal DNA amplification (see for example Lizardi, et al., Nat Genet, 19:225-32, 1998); (f) quantitative hybridization to the immobilized target locus (see for example Southern (1975) J. Mol. Biol. 98:503); and (g) loss of polymorphic markers unique to the targeted locus. For a detailed description of how one might assay for successfully genetically modified mammalian cells, see U.S. Pat. No. 6,586,251, and Valenzuela et al. (2003) Nature Biotechnology 21(6):652-659, each of which are incorporated herein by reference.

Use of Genetically Mutated and/or Modified Mammalian Cells

The mutated and/or modified mammalian cells generated by the method of the invention can be employed in any in vitro or in vivo assay. For example, the cells may be used for protein production, gene therapy, cell therapy, or in cell based assays such as drug discovery screening assays.

The genetically modified mammalian cells generated by the methods of the invention can also be used to generate non-human organisms carrying the genetic modification. The genetically modified mammalian cells can be used to generate non-human organisms by several different techniques including but not limited to (a) modified ES cells such as the frequently used mouse ES cells, which can be used to create genetically modified mice by standard blastocyst injection technology or aggregation techniques (see for example Robertson (1987) Practical Approach Series 254), tetraploid blastocyst injection (see Wang et al. (1997) Mech Dev, 62:137-45), or nuclear transfer and cloning (see Wakayama, et al. (1999) Proc Natl Acad Sci USA, 96:14984-9). ES cells derived from other organisms such as rat, rabbit, cat, dog, cow, sheep, goat, pig, horse, or monkey or other mammals; (b) modified protoplasts used to generate genetically modified plants (see for example U.S. Pat. Nos. 5,350,689 and 5,508,189); (c) nuclear transfer from modified mammalian cells to oocytes to generate cloned organisms with modified alleles (see for example Wakayama et al. Proc Natl Acad Sci USA, 96:14984-9); (d) cell-fusion to transfer the modified allele to another cell, including transfer of engineered chromosome(s), and uses of such cell(s) to generate organisms carrying the modified allele or engineered chromosome(s) (see Kuroiwa et al. (2000) Nat Biotechnol, 18:1086-1090).

Genetically Mutated and/or Modified Non-human Organisms

In one embodiment, the invention is directed to a transgenic animal which possesses a recombinant nucleic acid encoding a marker gene within its genome. Such a recombinant nucleic acid can comprise, for example, a nucleic acid encoding a marker gene (e.g., lacZ) which is operably linked to a promoter and/or enhancer from an endogenous gene. Detection of the marker gene can, for example, comprise staining a tissue sample obtained from a transgenic animal which expresses the marker gene, with a substance appropriate for detection of expression of the marker gene. Suitable marker genes and techniques for detection are described herein and/or are well known in the art.

One use of a transgenic animal having a marker gene is a method for testing an effect of an agent (e.g., a drug, a nucleic acid, a gene product, a targeting molecule) on a particular biological response. The method can comprise administering the agent to a transgenic animal (e.g., a mouse, including an embryo, a neonate, a juvenile, an adult) having a marker gene inserted in a gene of interest, and observing the effect of the agent on the biological activity associated with the gene of interest, as compared to the effect in a suitable control transgenic animal having the marker gene and maintained under identical conditions, but not administered the agent.

In one embodiment, the invention is drawn to a knockout animal in which the expression of a gene of interest within its genome has been interrupted. One use of a knockout animal in which the expression of a gene of interest within its genome has been interrupted is as an animal model system for diseases and conditions associated with the function of the knocked out gene of interest. Such a model system is also used for identifying therapeutic agents and/or treatments of the diseases and conditions. The present invention also relates to a method for identifying therapeutic agents for treatment of an individual diagnosed with a clinical disorder associated with a mutation in the gene of interest in which normal expression is altered or otherwise abnormal. The knockout animal is administered a candidate therapeutic agent and is then assayed for therapeutic effects resulting from the administration of the candidate therapeutic agent, as determined from the use of appropriate experimental controls. Therapeutic effects are indicated by a reduction or reversal of symptoms or amelioration of the general condition of the knockout animal. Screening of candidate therapeutic agents such as small molecules from molecular libraries, presently known drugs, and molecules for use in gene therapy, will identify therapeutic agents for treatment of a human patient diagnosed with a disorder similar to that of the animal model used. This model system can also be used for the identification of optimal methods of delivery and vectors for use in the gene therapy methods described above. This method can also be adapted to identify agents which prevent the development of a clinical disorder in an individual with a disorder associated with the gene of interest, for instance by administering the candidate agent to an asymptomatic knockout animal.

Other uses for genetically modified non-human organisms, especially transgenic and knockout organisms, for example transgenic and knockout mice, are familiar to skilled artisans.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Summary of Results of Gene Targeting Experiments Using a Ubiquitin Promoter to Drive Drug Resistance Gene Expression

Targeting vectors were constructed as described in, for example, U.S. Pat. No. 6,586,251, in the name of Regeneron Pharmaceuticals Inc. and Valenzuela, et al. Nature Biotechnology (2003) 21(6):652-659, each of which are incorporated herein by reference.

As is summarized in Table 2 below, in all tested cases, the use of a ubiquitin promoter to drive expression of a drug resistance gene increased the number of colonies surviving drug selection by an average of 6-fold over the use of the PGK promoter (see Column 7). The ubiquitin promoter's effect is strong enough to rescue some of the most difficult to target loci (for example, gene D and gene L, Column 7). In fact, in previous experiments that did not utilize a ubiquitin promoter driving expression of a drug resistance gene, all of the tested genes had extremely low targeting frequencies. It was only by utilizing the method of the invention that sufficient numbers of ES cell colonies were producced surviving drug selection to obtain correctly targeted clones.

In addition, there is a concomitant average 4fold increase in targeting efficiency (see Column 8). An increased targeting frequency is not necessarily expected as a result of an increase in the number of surviving colonies, as colonies can arise from both targeted gene modifications and non-targeted random insertions.

Taken together, these results demonstrate that the use of a ubiquitin promoter to drive expression of the drug resistance gene enhances productivity by reducing the number of colonies that need to be screened to obtain the desired correctly targeted clones.

TABLE 2

| | | 3 | 4 | 5 | | 7 | 8 |
|---|---|---|---|---|---|---|---|
| 1 | 2 | Colonies per | Clones | Targeted | 6 | Fold Increase | |
| Gene | Promoter | Electroporation | Screened | Clones | % Targeting | Colonies | % Targeting |

| | | | | | | | |
|---|---|---|---|---|---|---|---|
| T | PGK | 270 | 144 | 2 | 1.4 | | |
| T | Ubiquitin | 1804 | 576 | 19 | 3.3 | 7 | 2.4 |
| D | PGK | 20 | 0 | | | | |
| D | Ubiquitin | 313 | 288 | 0 | 0 | 16 | ND* |
| F | PGK | 42 | 40 | 1 | 2.5 | | |
| F | Ubiquitin | 291 | 288 | 47 | 16.3 | 7 | 6.5 |
| N | PGK | 32 | 32 | 0 | 0 | | |
| N | Ubiquitin | 96 | 96 | 1 | 1 | 3 | ND |
| P | PGK | 97 | 96 | 1 | 1 | | |
| P | Ubiquitin | 200 | 192 | 7 | 3.6 | 2 | 3.6 |
| 1R7 | PGK | 224 | 192 | 1 | 0.5 | | |
| 1R7 | Ubiquitin | 960 | 288 | 5 | 1.7 | 4 | 3.4 |
| 20 | PGK | 477 | 288 | 3 | 1 | | |
| 20 | Ubiquitin | 1436 | 288 | 6 | 2 | 3 | 2 |
| L | PGK | 73 | 0 | | | | |
| L | Ubiquitin | 354 | 288 | 0 | 0 | 5 | ND |
| E | PGK | 591 | 288 | 4 | 1.3 | | |
| E | Ubiquitin | 2370 | 288 | 19 | 6.1 | 4 | 5 |
| S | PGK | 411 | 288 | 0 | 0 | | |
| S | Ubiquitin | 2444 | 288 | 6 | 2.1 | 6 | >6 |

*ND = not determined

Average increase with ubiquitin promoter: Colonies, 6-fold; Targeting frequency, >4-fold

1. A method of generating embryonic stem (ES) cell colonies exhibiting drug resistance to a selection agent, comprising introducing into the ES cells an exogenous DNA comprising a ubiquitin promoter, and a drug resistance gene under control of the ubiquitin promoter.

2. The method of claim 1, wherein the ES cells are mammalian ES cells.

3. The method of claim 2, wherein the mammalian ES cells are mouse ES cells.

4. The method of claim 1, wherein the drug resistance gene encodes neomycin phosphotransferase.

5. The method of claim 1, wherein the drug resistance gene encodes hygromycin phosphotransferase.

6. The method of claim 1, wherein the drug resistance gene encodes puromycin acetyl transferase.

7. The method of claim 1, wherein the ubiquitin promoter is the ubiquitin C promoter.

8. The method of claim 7, wherein the ubiquitin promoter is a human, mouse, rat, or bacterial ubiquitin promoter.

9. A method of targeting a targeting vector into ES cells, comprising introducing into the ES cells a targeting vector comprising a drug resistance gene under control of a ubiquitin promoter.

10. The method of claim 9, wherein the ES cells are mammalian ES cells.

11. The method of claim 10, wherein the mammalian ES cells are mouse ES cells.

12. The method of claim 9, wherein the drug resistance gene encodes neomycin phosphotransferase.

13. The method of claim 9, wherein the drug resistance gene encodes hygromycin phosphotransferase.

14. The method of claim 9, wherein the drug resistance gene encodes puromycin acetyl transferase.

15. The method of claim 9, wherein the ubiquitin promoter is the ubiquitin C promoter.

16. The method of claim 15, wherein the ubiquitin promoter is a human, mouse, rat, or bacterial ubiquitin promoter.


SEQUENCE LISTING (3 sequences)

SEQ ID NO: 1 - DNA, 1212 bp, Homo sapien
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttggtgagtagcgggctgctgggctggccggggctttcgtggccgccgggccgctcggtgggacggaggcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctctggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 2 - DNA, 1837 bp, mus musculus
ccaggccgcgaagccgcagggcgcctgcgccaaggcccgctccggcctcagtgatcccagccgtgttttcgtgccgatcgtctcacgcgcgctgatccctccgcggagtcgcccgaggtcacagccctgccctcccacacaaagcccctcaatctctggacgccaccgtgaaacaactccgtgagagaggtaccttgatagttttagcctgtcgctttcgctgccgagactggacccggcgttacaaagtagtccctgaccgcattgcccgcggagggaccgcgcggaagggggggggcggggcttcggtgactatataaagagacgccgggcgtgccgcagctagttccgtggagactgcgagttccgtctgctgtgtgaggactgccgccaccaccgctggtgaggagaaagccgccgcacccggtcggggacgggaggctggaggcgagacggggcgagaggcagccccgcggcccagacgtttggtttccgtggcccgcgcggaccgcggctgccccgaggcagaggactgggcggcaagatggcggccagatggaagcctgagggggaagacgcggggctctgacgcgcaggacgaggttgggggaggaaaaaggcccgcgaggccgctgccctccggttaagccggggacgtcggagactgtggggtggggactgaattagggttgcgcgccgtaggagcctctgctgtgagagccgtggatattgggctggcccgagaggtcgattggcccggcgttcgtccgttcgtttgctgaaagacggaagtgcgatcgagaccggaagggggttgggcggcggttcagcctgcctggcctgccgccccctgtgacgtcgcgggttgcgtggcctcctaatggatagtgacgtcactatcttgactttagctttccctcggttgtaggacagggtttgggtctcggcctccggtagcctctccagagtaaacaggaaccggaaattcagaggggaaatgtgagccattcttgtcctgtttcgttttaagaatgtcgctgtacaactatgactactgaaacttttggggggggggttcgagacggtttctctatgtagtcctggctgtcccgcatctcactctgtagatcaggctggcctcgaacccagaaatcctcctgcctctgcctcccaagtgttgggacgaaaggcaccaccattgtcctgcgacaagggtgtttttttttaaactgtcaaaatctctgcctctaccaccccatgtgatgaggtccaaggccagtaccaccactccagactaattttaatcgttcagacaaaagtttggtgttcttttgggggaaggagagttgaggcaggattacactgtctctggctgaccttccagttagagatctgcccacctcagtgtccccagtgctgaggtcagcgataggcatgggctcagacttagttttgcagtagtaacttgctatattaccattctgaaactgaatccgggactgctgtggtttcataacctcccagaggtcaggcttttctgcaaactgttcaaatagacagaaattgactttcagctgttggtatactgaagtctccatcctgtaaatttggtaatacaaaaagactcaccatgccgaggtttcttaactttgttagtcaacaatcttattttcttgatggtttttcggggtggggggattggattcaagacagaatctgtgtagatagaccttgctatttagacttatagcatccagttgacaaatgttgatgccatcccacaaatatttgtgtcattcctgacctgtgaattgttttgtatattttgtgacagacg

SEQ ID NO: 3 - DNA, 2133 bp, Rattus norvegicus
ccggctcgcttagccacagtgcgcctgcgccgaggtccgctccggctgcagtgatccctgccgggttttagcgccgatcacgtcacaggcgctgatccctccgcggggtcgcgcaaggtcgcagtcctgcccctccacacaaagcccctcactctctggccttcgcagtggaacaaccccatgaagcgatcttaatggttttagcctgtcgcttccattgcagagattggaccgggcattaaaaagtagtccccaaccgcatttccgcggaggaatccaggggtgggcggggctcccgatgactatataaagagacgcctcgcgtcccgcagctagttccgtggagaccgccagttccgtctctactcttttgtgaggactgcagccaacaccgctggtgaggagaaagccgccacacccggcgggggcggggaggcgggagggggcgagagacagcccgcggcccggacgtttggtttccgtggcccgcgaggacggctgctgtcctgaggccgaggaccgggcggcaagatggcggccaaatggaagcctgagggggaagacgcgcggtcctgacgcgctggaccaggttgggggaagcaggcccgcgaggccgcttgccgccggttaagccgggaacgtcagaggcttgggttgggaactgaattcggttggcgcgctgtaggagcctctgatgtgagaactgtgggtattagacgggagggagatgtcggttatccggaattcgtcggttgctaaatacaggagtgcgattgggaagggagggggagggtggcggtccagcctagcctatcgcccctgtgacgtcgcggtttgtgtggtctcacagtgtatagtgacgatggctttagctttccctcagttgtaggaccgggtttgggtctcggcctcgggtagcctctccagagtaaacaggaaccggaaattcagagggggaacgtgagcctgacttacattgagtctttccttgtctcgctacacggatatggctgcggaaactatttattagacaagggtgtttttttttttaagatttatttcttcaggggttggggatttagctcagtggtagagcgcttgcctaggaagcgcaaggccctgggttcggtccccagctccggaaaaaaagaaccaaaaaaaaaaaagatttatttcttcagacacaccagaagaggtgtgatcagatctcattacaaatggttgttagccaccatatggttgccgggaattgaactcgggacctctggaagagcagtcagtgctattaatcactgagtcatcttaccagcctgacaagggttttaaactttaaaaaaatctctgcctctaccacccatgtgatgaggtccaaggccagtgccacttaccccagactacttttttttttaatagagtttattcagggcatgggaaggggagttgaggagggagtaaagctggtcggaacatgtggagagagagcagggaggagaatggggagagaagggacagatggggtgaaagtaagaggttaagaggataagagtaagagtaaactactgtttaattgttcagacaaaagcttgggggaaggagagttgaggcaggatctcactgtctcaggctgacctcccaattcacgaaatctgcccacctcagtgtccccagtgctgggaggaaagatatgaacatagttcagttttgtagtagtaactggctctatctaccagtcgggacctgaatccgggatttccaactattgccttctaagctcttgagtttctgacttctttggagtgggtcctcctaaacccccctcccccaaggtgaaatgtgggtcaggcttttctccaaactgcaaatagtcagggactgactttcagcagttccgtactgaacgctccaccctgccaacttggtaatagacaatgcctaatattcataactttgttccatcaagacttttattttcttgttggttttagggggtaaaattcaaaacagtctgtgtagctatttagactaaactactagccacctcacagatccaacggttggtggcactctaaagaatttgtgtcattcctgacctgttttttggttttttgttttttgtttttttttccatagaca